(19) United States
(12) Patent Application Publication
(10) Pub. No.: US 2023/0116844 A1
(43) Pub. Date: Apr. 13, 2023

(54) COMPOSITIONS AND METHODS FOR TESTING ADVERSE SKIN EVENTS

(71) Applicant: NOVOCURE GMBH, Root D4 (CH)

(72) Inventor: AVISHAI NAHSHONI, Haifa (IL)

(21) Appl. No.: 17/953,823

(22) Filed: Sep. 27, 2022

Related U.S. Application Data
(60) Provisional application No. 63/248,874, filed on Sep. 27, 2021.

(51) Int. Cl. A61B 5/00; A61F 13/02; A61N 1/04; A61N 1/36

(52) U.S. Cl. CPC A61B 5/6833; A61B 5/411; A61F 13/0253; A61N 1/0492; A61N 1/36014; A61F 2013/00676

(57) ABSTRACT

Disclosed are apparatuses comprising at least one electrode element having a skin-facing surface; a skin contact layer comprising an adhesive composite, and an adhesive tape or bandage. Disclosed are methods comprising positioning one or more electrode assemblies on a target site of a subject, the one or more electrode assemblies comprising at least one electrode element having a skin-facing surface; a skin contact layer comprising a conductive adhesive composite, and an adhesive tape or bandage, wherein the at least one electrode element is electrically coupled to the skin contact layer. In some aspects, the methods further comprise removing at least one of the one or more electrode assemblies from the target site of the subject after a period of time. In some aspects, after removal of the one or more electrode assemblies, the target site is evaluated for an adverse event.

### CROSS-REFERENCE TO RELATED APPLICATIONS

This application claims the benefit of U.S. Provisional Pat. Application No. 63/248,874, filed on Sep. 27, 2021, which is incorporated by reference herein in its entirety.

### BRIEF SUMMARY

Disclosed are apparatuses comprising at least one electrode element having a skin-facing surface; a skin contact layer comprising an adhesive composite, and an adhesive tape or bandage.

Disclosed are methods comprising positioning one or more electrode assemblies on a target site of a subject, the one or more electrode assemblies comprising at least one electrode element having a skin-facing surface; a skin contact layer comprising a conductive adhesive composite, and an adhesive tape or bandage, wherein the at least one electrode element is electrically coupled to the skin contact layer. In some aspects, the methods further comprise removing at least one of the one or more electrode assemblies from the target site of the subject after a period of time. In some aspects, after removal of the one or more electrode assemblies, the target site is evaluated for an adverse event, such as, for example, a skin irritation.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 illustrates an example Study Schema. * indicates not to be done if combination X (described below) is selected in part 1.

FIG. 2 illustrates an array layout on a subject’s body to compare different combinations of adhesives and conductive materials.

FIG. 3 illustrates an array layout on a subject’s body to evaluate the effect of prophylactic topical steroids / PDE4 inhibitors / calcineurin inhibitors / skin barriers.

FIG. 4 illustrates an array layout on a subject’s body for determining the best frequency and method of changing arrays.

### DETAILED DESCRIPTION

The disclosed method and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an electrode” includes a plurality of such electrodes, reference to “the apparatus” is a reference to one or more apparatuses and equivalents thereof known to those skilled in the art, and so forth.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directions. In some aspects, alternating electric fields can be delivered through two pairs of transducer arrays that generate perpendicular fields within the target area. For example, for the Optune™ system (an alternating electric fields delivery system) one pair of electrodes is located to the left and right (LR) of the target area, and the other pair of electrodes is located anterior and posterior (AP) to the target area. Cycling the field between these two directions (i.e., LR and AP) ensures that a maximal range of cell orientations is targeted.

In-vivo and in-vitro studies show that the efficacy of alternating electric fields therapy increases as the intensity of the electrical field increases. Therefore, optimizing array placement on the patient to increase the intensity in the desired region can be performed with the Optune system. Array placement optimization may be performed by “rule of thumb” (e.g., placing the arrays on the chest as close to the desired region of the target site (e.g., lungs) as possible), measurements describing the geometry of the patient’s body, or body dimensions. Measurements used as input may be derived from imaging data. Imaging data is intended to include any type of visual data. In certain implementations, image data may include 3D data obtained from or generated by a 3D scanner (e.g., point cloud data). Optimization can rely on an understanding of how the electrical field distributes within the body (for example, within the lung) as a function of the positions of the array and, in some aspects, take account for variations in the electrical property distributions within the target site (e.g., infected cell) of different patients.

The term “subject” refers to the target of administration, e.g. an animal. Thus, the subject of the disclosed methods can be a vertebrate, such as a mammal. For example, the subject can be a human. The term does not denote a particular age or sex. “Subject” can be used interchangeably with “individual” or “patient.” For example, the subject of administration can mean the recipient of the alternating electrical field.

As used herein, a “target site” is a specific site or location within or present on a subject or patient. For example, a “target site” can refer to a specific location on the skin of a subject. For example, the target site can be on the head, chest, back, arm, or leg.

As used herein, the terms “administering”, “administration”, and “applying” refer to any method of providing an agent, such as a skin protectant agent, to a subject. Such methods are well known to those skilled in the art and include, but are not limited to: oral administration, transdermal administration, administration by inhalation, nasal administration, topical administration, intravaginal administration, ophthalmic administration, intramural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, applying alternating electrical fields or exposing a subject to alternating electrical fields means administering alternating electrical fields to the subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The apparatuses disclosed herein can be used for applying alternating electric fields and the methods disclosed herein comprise alternating electric fields. In some aspects, the alternating electric fields used in the methods disclosed herein are tumor-treating fields (TTFields). In some aspects, the alternating electric fields can vary dependent on the type of cell or condition to which the alternating electric fields are applied. In some aspects, the alternating electric fields can be applied through one or more electrodes placed on the subject’s body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric fields can then be applied and can alternate between the front and back electrodes and then to the side to side electrodes.

In some aspects, the frequency of the alternating electric fields can be 150 kHz. The frequency of the alternating electric fields can also be, but is not limited to, about 150 kHz, about 200 kHz, between 50 and 500 kHz, between 100 and 500 kHz, between 25 kHz and 1 MHz, between 50 kHz and 1 MHz, between 50 and 190 kHz, between 25 and 190 kHz, or between 210 and 400 kHz. In some aspects, the frequency of the alternating electric fields can be electric fields at 50 kHz, 100 kHz, 150 kHz, 200 kHz, 300 kHz, 400 kHz, 500 kHz, 1 MHz, or any frequency between.

In some aspects, the frequency of the alternating electric fields is from about 150 kHz to about 300 kHz, from about 100 kHz to about 300 kHz from about 200 kHz to about 400 kHz, from about 250 kHz to about 350 kHz, or may be around 300 kHz.

In some aspects, the field strength of the alternating electric fields can be between 1 and 5 V/cm RMS. In some aspects, different field strengths can be used (e.g., between 0.1 and 10 V/cm). In some aspects, the field strength can be 1.75 V/cm RMS. In some embodiments the field strength is at least 1 V/cm. In some aspects, combinations of field strengths are applied, for example combining two or more frequencies at the same time, and/or applying two or more frequencies at different times.

In some aspects, the alternating electric fields are applied at a frequency of between 250 kHz and 350 kHz. In some aspects, the alternating electric fields are applied at a frequency between 50 and 190 kHz. In some aspects, the alternating electric fields are applied at a frequency between 210 and 400 kHz. In some aspects, the alternating electric fields have a field strength of at least 1 V/cm RMS. In some aspects, the alternating electric fields have a frequency between 50 and 190 kHz. In some aspects, the alternating electric fields have a frequency between 210 and 400 kHz. In some aspects, the alternating electric fields have a field strength of at least 1 V/cm RMS. In some aspects, the alternating electric fields have a field strength between 1 and 4 V/cm RMS.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

On occasion, some subjects undergoing TTFields treatment have reported skin irritation beneath the arrays. Since survival benefit from TTFields increases with duration-of-use, prevention and management of skin irritation is important to maximize patient adherence to the recommended duration of the treatment plan. The devices and methods of use disclosed herein are directed to better outcomes with respect to skin irritation, which in turn will benefit patient outcomes.

### C. Devices

Disclosed are devices that comprise or utilize one or more of the apparatuses disclosed herein. Disclosed herein are devices for use in the disclosed methods.

In some aspects, the disclosed devices can be any device for electrotherapeutic treatment. Generally, the device can be a portable, battery or power supply operated device that produces alternating electrical fields within the body by means of transducer arrays or other electrodes. The device can comprise an electrical field generator and one or more electrode (e.g., transducer) arrays, each comprising a plurality of electrodes. In some aspects, the device can comprise an apparatus comprising at least one electrode element having a skin-facing surface; a skin contact layer comprising an adhesive composite, and an adhesive tape or bandage. The device can be configured to generate tumor treating fields (TTFields) (e.g., at 150 kHz or 200 kHz) via the electrical field generator and deliver the TTFields to an area of the body through the one or more electrode arrays. The electrical field generator can be a battery and/or power supply operated device.

The electrical field generator can comprise a processor in communication with a signal generator. The electrical field generator can comprise control software configured for controlling the performance of the processor and the signal generator. Although it can be within the electrical field generator, it is contemplated that the processor and/or control software can be provided separately from the electrical field generator, provided the processor is communicatively coupled to the signal generator and configured to execute the control software.

The signal generator can generate one or more electric signals in the shape of waveforms or trains of pulses. The signal generator can be configured to generate an alternating voltage waveform at frequencies in the range from about 50 KHz to about 1 MHz (preferably from about 100 kHz to about 300 kHz) (e.g., the TTFields). The voltages are such that the electrical field intensity in tissue to be treated is typically in the range of about 0.1 V/cm to about 10 V/cm.

One or more outputs of the electrical field generator can be coupled to one or more conductive leads that are attached at one end thereof to the signal generator. The opposite ends of the conductive leads are connected to the one or more electrode arrays that are activated by the electric signals (e.g., waveforms). The conductive leads can comprise standard isolated conductors with a flexible metal shield and can be grounded to prevent the spread of the electrical field generated by the conductive leads. The one or more outputs can be operated sequentially. Output parameters of the signal generator can comprise, for example, an intensity of the field, a frequency of the waves (e.g., treatment frequency), a maximum allowable temperature of the one or more electrode arrays, and/or combinations thereof. In some aspects, a temperature sensor can be associated with each electrode array. Once a temperature sensor measures a temperature above a threshold, current to the electrode array associated with said temperature sensor can be stopped until a second, lower threshold temperature is sensed. The output parameters can be set and/or determined by the control software in conjunction with the processor. After determining a desired (e.g., optimal) treatment frequency, the control software can cause the processor to send a control signal to the signal generator that causes the signal generator to output the desired treatment frequency to the one or more electrode arrays.

The one or more electrode elements can be configured in the disclosed apparatuses in a variety of shapes and positions so as to generate an electrical field of the desired configuration, direction and intensity at a target site (referred to herein also as a “target volume” or a “target region”) so as to focus treatment. Optionally, the one or more electrode arrays can be configured to deliver two perpendicular field directions through the volume of interest.

In some embodiments an apparatus comprises at least one electrode element having a skin-facing surface; a skin contact layer comprising an adhesive composite, and an adhesive tape or bandage. In some embodiments the at least one electrode element is electrically coupled to the skin contact layer. In some embodiments the skin contact layer is disposed on the skin-facing surface of the at least one electrode. Such apparatus may be used for applying alternating electric fields. In some embodiments the at least one electrode comprises the skin contact layer.

The adhesive composite used in connection with the apparatus may be a conductive adhesive composite. For example, a conductive adhesive composite may include a dielectric material; and a conductive component dispersed within the dielectric material. In some embodiments the adhesive composite comprises an acrylic polymer. In some embodiments the adhesive composite comprises a silicone polymer. In some embodiments the adhesive composite comprises an acrylic polymer and a silicone polymer. In some embodiments the adhesive composite comprises a hydrogel. In some embodiments the adhesive composite comprises an acrylic polymer and a hydrogel. In some embodiments the adhesive composite comprises a silicone polymer and a hydrogel. In some embodiments the adhesive composite comprises an acrylic polymer, a silicone polymer and a hydrogel. In another example, a conductive adhesive composite may include a dielectric material and conductive particles dispersed within the dielectric material. In some embodiments the adhesive composite comprises an acrylic polymer and conductive particles dispersed therein. In some embodiments the adhesive composite comprises a silicone polymer and conductive particles dispersed therein. In some embodiments the adhesive composite comprises an acrylic polymer, a silicone polymer, and conductive particles dispersed therein. The conductive adhesive composite may be a dry carbon/salt adhesive. In some embodiments the adhesive composite comprises a hydrogel. In some embodiments the adhesive composite comprises an acrylic polymer and/or a silicone polymer, a hydrogel, and conductive particles dispersed therein. In other embodiments the adhesive composite excludes a hydrogel. Conductive particles may include, for example, carbon particles, such as graphite powder, carbon flakes, carbon granules, carbon fibers, carbon nanotubes, carbon nanowires, or carbon black powder. Optionally, the conductive particles comprise a plurality of groups of conductive particles. In some embodiments, at least a portion of the conductive particles define a conductive pathway through a thickness of the conductive adhesive composite. In further aspects, the conductive adhesive composite further comprises a polar material (e.g., a polar salt). The polar salt may be a quaternary ammonium salt, such as a tetra alkyl ammonium salt.

In some embodiments, the conductive adhesive composite may be a non-hydrogel biocompatible conductive adhesive such as the developmental product FLX068983 -FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® EL-8006 electrically conductive adhesive composition manufactured and sold by Adhesives Research, Inc. (Glen Rock, PA, USA).

The adhesive tape or bandage used in connection with an apparatus disclosed herein may be used to secure an electrode array to the subject’s body. The adhesive tape or bandage may comprise a silicone adhesive, an acrylic adhesive, or a combination thereof. For example, a suitable acrylic adhesive tape may be a Polyethylene Foam Single Coated Medical Tape 1773 (3M, St. Paul, MN, USA). For example, a suitable silicone adhesive tape may be a 2480 Single-Coated Medical Nonwoven Tape with Hi-Tack Silicone Adhesive on Liner (3M, St. Paul, MN, USA).

### D. Methods

In some embodiments a method comprises positioning one or more electrode assemblies on a target site of a subject, the one or more electrode assemblies comprising: at least one electrode element having a skin-facing surface; a skin contact layer comprising a conductive adhesive composite, and an adhesive tape or bandage, wherein the at least one electrode element is electrically coupled to the skin contact layer. In some embodiments a method may further comprise removing at least one of the one or more electrode assemblies from the target site of the subject after a period of time, for example a period of time of between 5 to 60 minutes, 1 to 24 hours, 1 to 7 days, 1 to 4 weeks, or 1 to 6 months.

In some embodiments of a method, the removing step comprises applying one or more of mineral oil, hexamethyldisiloxane, and a wipe comprising isopropyl alcohol, hydrotreated heavy naphtha, (2-methoxymethylethoxy)propanol. An example of a suitable hexamethyldisiloxane product is APPEEL™ sterile medical adhesive remover. An example of a suitable wipe comprising isopropyl alcohol, hydrotreated heavy naptha, and (2-methoxymethylethoxy)propanol is Uni-Solve™ wipe sold by Smith & Nephew.

In some embodiments a method may further comprise applying an alternating voltage between at least two electrode assemblies, thereby generating an alternating electric field, wherein applying the alternating voltage occurs prior to removing at least one of the electrode assemblies. In some embodiments a method may further comprise applying a skin protectant agent to the target site after removing at least one of the one or more electrode assemblies.

Suitable skin protectants for use with the methods disclosed herein include one or more of a steroid, an alcohol, and a siloxane copolymer. In some embodiments the steroid may comprise betamethasone. In some embodiments the alcohol may comprise isopropyl alcohol. In some embodiments the alcohol may be applied by a wipe. In some embodiments the siloxane copolymer may comprise hexamethylsiloxane/acrylate copolymer.

In some embodiments of a method the adhesive composite comprises a hydrogel. In other embodiments the adhesive composite excludes a hydrogel. In some embodiments a hydrogel is not present between the target site and the skin-facing surface of the one or more electrodes. In some embodiments the adhesive composite comprises a dielectric material and a conductive component dispersed within the dielectric material. In some embodiments the adhesive composite comprises an acrylic polymer. In some embodiments the adhesive composite comprises a silicone polymer. In some embodiments the adhesive composite comprises an acrylic polymer and a silicone polymer. In some embodiments the adhesive composite comprises a hydrogel. In some embodiments the adhesive composite comprises an acrylic polymer and a hydrogel. In some embodiments the adhesive composite comprises a silicone polymer and a hydrogel. In some embodiments the adhesive composite comprises an acrylic polymer, a silicone polymer and a hydrogel. In another example, a conductive adhesive composite may include a dielectric material and conductive particles dispersed within the dielectric material. In some embodiments the adhesive composite comprises an acrylic polymer and conductive particles dispersed therein. In some embodiments the adhesive composite comprises a silicone polymer and conductive particles dispersed therein. In some embodiments the adhesive composite comprises an acrylic polymer, a silicone polymer, and conductive particles dispersed therein. The conductive adhesive composite may be a dry carbon/salt adhesive. In some embodiments the adhesive composite comprises an acrylic polymer and/or a silicone polymer, a hydrogel, and conductive particles dispersed therein. Conductive particles may include, for example, carbon particles, such as graphite powder, carbon flakes, carbon granules, carbon fibers, carbon nanotubes, carbon nanowires, or carbon black powder. Optionally, the conductive particles comprise a plurality of groups of conductive particles. In some embodiments, at least a portion of the conductive particles define a conductive pathway through a thickness of the conductive adhesive composite. The conductive adhesive composite may further comprise a polar material (e.g., a polar salt). The polar salt may be a quaternary ammonium salt, such as a tetra alkyl ammonium salt. In some embodiments the conductive adhesive composite of the one or more electrode assemblies has a thickness ranging from about 30 µm to about 2000 µm. In some embodiments the conductive adhesive composite of one electrode assembly can be different from the conductive adhesive composite of another electrode assembly. Examples of conductive adhesive composites are discussed above. In some embodiments the target site does not comprise hair.

In some embodiments a method further comprises, after removing the at least one of the one or more electrode assemblies from the target site, re-positioning the at least one of the one or more electrode assemblies on the target site. In other embodiments a method further comprises positioning a second one or more electrode assembly on the target site, after removing the at least one of the one or more electrode assemblies from the target site. Methods of the invention may further include, after re-positioning, removing the one or more electrode assemblies (or the second one or more electrode assemblies) after a second period of time. For example, a suitable second period of time may be between 5 to 60 minutes, 1 to 24 hours, 1 to 7 days, 1 to 4 weeks, or 1 to 6 months. In some embodiments a method may further comprise applying a second alternating voltage between at least two electrode assemblies, thereby generating an alternating electric field, wherein applying the second alternating voltage occurs after re-positioning the at least one of the electrode assemblies and prior to removing the one or more electrode assemblies after a second period of time.

In some embodiments a method may further comprise determining the ease of removing the one or more electrode assemblies.

In some embodiments a method may further comprise, after removal of the one or more electrode assemblies, evaluating the target site for an adverse event. For example, such adverse event may be contact dermatitis, pruritus, erosions, ulcers, folliculitis, or xerosis.

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed apparatuses. The kits can further comprise a skin protective agent. Optionally, the kit can further comprise instructions for carrying out one or more of the methods disclosed herein.

### F. Examples

Evaluation of skin irritation events caused by transducer arrays in healthy volunteers.

Study aim: Determine which products cause the least skin irritation and how they should be used.

Study type: Prospective, unicenter, open label, single arm.

Study Population: Healthy male and female volunteers.

Objectives: 1) To determine a favorable adhesive (for example, silicone versuspolymers, acrylate acrylic polymers, etc.) in terms of adverse events and contact with the skin irritation. 2) To determine a favorable conductive material. 3) To assess whether OMNI-WAVE and/or ARcare EL8006 without additional adhesives is comfortable and well-tolerated. 4) To investigate skin protectant agents / skin barriers / topical steroids / PDE4 inhibitors / calcineurin inhibitors (such as siloxane copolymers, alcohols, betamethasone, “Smith and Nephew Skin-Prep wipes” and “Convatec Sensi-Care Sting Free Skin Barrier”) for their potential role in preventing or treating skin irritation. 5) To determine the favorable frequency of changing arrays. 6) To compare adhesive removal with using one or more of: baby oil and adhesive removers (such as Appeel sterile / Smith & Nephew Uni-Solve Adhesive Remover Wipes / Silmed medical adhesive remover (spray), etc.).

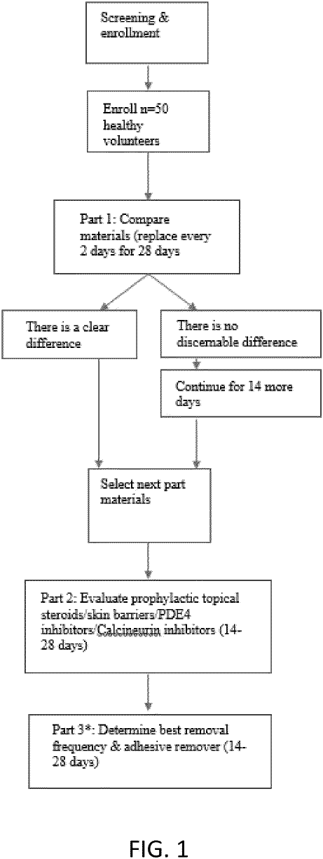

FIG. 1 illustrates an example Study Schema.

Statistical considerations: There is no formal statistical hypothesis. A sample size of up to 50 subjects is expected to provide the required data.

Estimated duration of study: 2 months.

Duration of participation: Total study duration for each healthy volunteer is approximately 4-10 weeks.

Inclusion/ exclusion criteria: Inclusion criteria includes healthy male and female volunteers ≥18 years of age; and signed informed consent form for the study protocol. Exclusion criteria includes any active acute or chronic disease judged to be clinically significant by the investigator; allergies to the materials examined; a positive history of atopy (allergic rhinitis, asthma, atopic dermatitis); any active acute or chronic skin disease judged to be clinically significant by the investigator; pregnancy or breastfeeding. Women of childbearing potential must have a negative urine test and must use effective contraception method during the entire period of the study; sunburn, tattoo or other skin damage in tested sites* (*For comparability and standardization, it is important to always use (if possible) the same anatomical site, preferably the upper back); excessive sun exposure/tanning in the last 3 weeks; use of topical steroids on tested sites in the last week; use of systemic corticosteroids 2 weeks before and during the trial

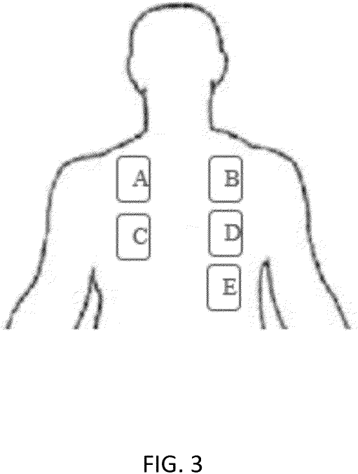

FIG. 3 illustrates an array layout on a subject’s body for Part 2.

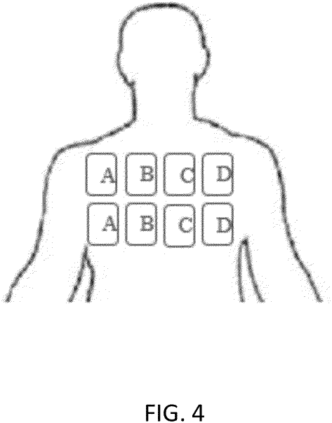

FIG. 4 illustrates an array layout on a subject’s body of Part 3.

Study description: The most suitable adhesives and conductive materials will be investigated along with their best ways methods of use. Up to 2050 healthy volunteers are expected to be enrolled in the study.

Parameters to be collected include background parameters and comfort of use parameters. Background parameters: Gender, Age, BMI, Outdoors activities, Hairiness (required shave in tested sites). Comfort of use parameters: Ease of arrays application, general comfort, durability of adhesion to the skin, ease of array removal.

Examined adverse events are contact dermatitis*, pruritus**, erosions, ulcers, folliculitis, xerosis. *Visual grading of allergic contact dermatitis (ACD) according to the International Contact Dermatitis Research Group method of Table 1. **Daily diary of itch NRS (numeric rating scale) incorporated in the volunteer questionnaire.

TABLE 1

| | |
|---|---|
| Negative reaction | - |
| Doubtful reaction: faint macular erythema only | ? |
| Weak positive reaction: non-vesicular with erythema, infiltration, possibly papules | + |
| Strong positive reaction: vesicular, erythema, infiltration, papules | ++ |
| Extreme positive reaction: bullous or ulcerative reaction | +++ |
| Irritant reaction: pustules as well as patchy follicular or homogeneous erythema without infiltration | IR |

Part 1 — Compare different combinations of adhesives and conductive materials: A — Acrylic Adhesive with hydrogel (6X6 cm); B — Acrylic adhesive with OMNI-WAVE (6X6 cm); C — Silicone adhesive with hydrogel (6X6 cm); D - Silicone adhesive with OMNI-WAVE (6X6 cm); X — OMNI-WAVE without additional adhesives, wrapped with gauze bandage (10X15 cm); Y (control) — Acrylic adhesive alone (3X6 cm); Z (control) -Silicone adhesive alone (3X6 cm)

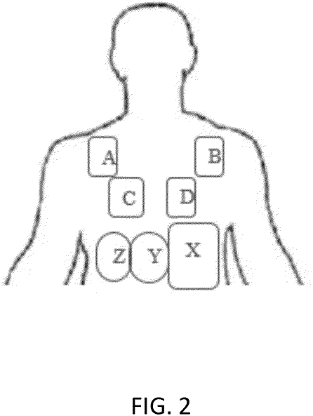

FIG. 2 illustrates an array layout on a subject’s body for Part 1.

The procedure involves the following steps: 1) Eligible healthy subjects will sign informed consent form; 2) Background parameters will be collected from each volunteer; 3) Hair in tested sites will be shaved; 4) Wash with antibacterial soap; 5) DSS Investigator will apply the different arrays on the subject’s back, as illustrated above, and draw a line around the outline; 6) Unidentified back photos will be taken; 7) Subject will go home with the arrays on and return to the clinic every 2 days for array removal, examination and replacement; 8) Each transducer array will be visually inspected prior to its removal for adhesion durability; 9) Subject and DSS Investigator will remove the transducer arrays using baby oil; 10) Unidentified back photos will be taken upon each removal; 11) Adverse events will be collected; 12)During the study period, subjects may only shower before the new arrays are applied; 13) Subject will complete a daily diary (volunteer questionnaire & activities) during the study period; 14) Subject will complete a questionnaire concerning comfort of use of the arrays and their removal; 15) DSS Investigator will complete a questionnaire concerning arrays application and removal.

In the same group of patients (if possible) — per the selected combination of adhesive and conductive material: Part 2 — Evaluate the effect of prophylactic topical steroids / PDE4 inhibitors / calcineurin inhibitors / skin barriers: A — apply array over “Smith and Nephew Skin-Prep wipes”; B — apply array over “Convatec Sensi-Care Sting Free Skin Barrier”; C — apply array over Betacorten solution; D — apply array over Betacorten solution & “Smith and Nephew Skin-Prep wipes”; E — apply array over Betacorten solution & “Convatec Sensi-Care Sting Free Skin Barrier.”

FIG. 3 illustrates an array layout on a subject’s body for Part 2. 1— Hair in tested sites will be shaved. 2— Wash with antibacterial soap. 3— DSS Investigator will apply 5 arrays (of chosen adhesive and/or conductive material) on the subject’s upper back (on healthy skin) and draw a line around the outline. 4— Each time, the arrays will be applied over the previously stated materials prophylactic topicals (skin protectant agents / skin barriers / topical steroids / PDE4 inhibitors / calcineurin inhibitors). 5— Unidentified back photos will be taken. 6— Subject will go home with the arrays on and return to the clinic every 2 days for array removal, examination and replacement (over the same prophylactic topicals). 7— Each transducer array will be visually inspected prior to its removal for adhesion durability. 8— Subject and DSS Investigator will remove the transducer arrays using the chosen adhesive remover in part 2 baby oil. 9— Unidentified back photos will be taken upon each removal. 10 — Adverse events: Skin irritation evaluations will be collected. 11— During the study period, subjects may only shower before the new arrays are applied. 12— Subject will complete a daily diary (volunteer questionnaire & activities) during the study period. 13— Subject will complete a questionnaire concerning comfort of use of the arrays and their removal. 14— DSS Investigator will complete a questionnaire concerning arrays application and removal.

Part 3 (not to be done if option X is selected in part 1) - Determine the best frequency and method of changing arrays: A — remove with baby oil; B — remove with Appeel sterile; C — remove with Smith & Nephew Uni-Solve Adhesive Remover Wipes; D — remove with Silmed medical adhesive remover spray. * Upper row — replace every 3 days, lower row - replace daily. ** Each arrays' size is 6X6 cm.

FIG. 4 illustrates an array layout on a subject’s body for Part 3. 1— Hair in tested sites will be shaved. 2— Wash with antibacterial soap. 3— DSS Investigator will apply 8 arrays (of chosen adhesive and conductive material) on the subject’s upper back (on healthy skin) and draw a line around the outline. 4— Unidentified back photos will be taken. 5— Subject will go home with the arrays on and return to the clinic every day. 6— 4 arrays will be replaced daily in the clinic - A - D lower row. 7— 4 arrays will be replaced every 3 days in the clinic — A — D upper row. 8— Each transducer array will be visually inspected prior to its removal for adhesion durability. 9— Unidentified back photos will be taken upon each removal. 10— Adverse events will be collected. 11— During the study period, subjects may only shower before the new arrays are applied. 12— Subject will complete a daily diary (volunteer questionnaire & activities) during the study period. 13— Subject will complete a questionnaire concerning comfort of use of the arrays and their removal. 14— DSS Investigator will complete a questionnaire concerning arrays application and removal.

Volunteers’ questionnaire is as follows:

Answer separately for each of the applications (A, B, C, D, X, Y, Z) with respect to the test area of the skin:

1. During the last day, how much did your skin itch, sting, or ache in the area where the material was applied?

| | | | | | | | | | |
|---|---|---|---|---|---|---|---|---|---|
| 1 | 2 | 3 | 4 | 5 | 6 | 7 | 8 | 9 | 10 |
| Not at all | | | | | | | | | Extremely |
| 2. On the last day, what was the maximum amount of itching or pain you experienced in the area you applied the material? | | | | | | | | | |
| 1 | 2 | 3 | 4 | 5 | 6 | 7 | 8 | 9 | 10 |
| Not at all | | | | | | | | | Unbearable |
| 3. On the last day, how comfortable was the material being tested? | | | | | | | | | |
| 1 | 2 | 3 | 4 | 5 | 6 | 7 | 8 | 9 | 10 |
| Not at all | | | | | | | | | Very comfortable |
| Ease of material application: | | | | | | | | | |
| 1 | 2 | 3 | 4 | 5 | 6 | 7 | 8 | 9 | 10 |
| Not easy at all | | | | | | | | | Very easy |
| Visually assess the tested material skin adhesion durability: | | | | | | | | | |
| 1 | 2 | 3 | 4 | 5 | 6 | 7 | 8 | 9 | 10 |
| Not durable at all | | | | | | | | | Very durable |
| Ease of material removal: | | | | | | | | | |
| 1 | 2 | 3 | 4 | 5 | 6 | 7 | 8 | 9 | 10 |
| Not easy at all | | | | | | | | | Very easy |

Those skilled in the art will recognize, or be able to ascertain using no more than routine experimentation, many equivalents to the specific embodiments of the method and compositions described herein. Such equivalents are intended to be encompassed by the following claims.

### G. Illustrative Embodiments

Illustrative Embodiment 1. An apparatus comprising: at least one electrode element having a skin-facing surface; a skin contact layer comprising an adhesive composite; and an adhesive tape or bandage.

Illustrative Embodiment 2. The apparatus of Illustrative Embodiment 1, wherein the at least one electrode element is electrically coupled to the skin contact layer.

Illustrative Embodiment 3. The apparatus of Illustrative Embodiment 1, wherein the apparatus is for applying alternating electric fields.

Illustrative Embodiment 4. The apparatus of any one of Illustrative Embodiments 1-3, wherein the adhesive composite is a conductive adhesive composite.

Illustrative Embodiment 5. The apparatus of Illustrative Embodiment 1, wherein the adhesive composite is a conductive adhesive composite comprising: a dielectric material; and conductive particles dispersed within the dielectric material.

Illustrative Embodiment 6. The apparatus of Illustrative Embodiment 5, wherein at least a portion of the conductive particles define a conductive pathway through a thickness of the conductive adhesive composite.

Illustrative Embodiment 7. The apparatus of any one of Illustrative Embodiments 5-6, wherein the conductive adhesive composite is a dry carbon/salt adhesive.

Illustrative Embodiment 8. The apparatus of any one of Illustrative Embodiments 5-7, wherein the dielectric material is an acrylic adhesive or a silicone adhesive, or a combination thereof.

Illustrative Embodiment 9. The apparatus of any one of the preceding Illustrative Embodiments, wherein the adhesive tape or bandage comprises a silicone adhesive, an acrylic adhesive, or a combination thereof.

Illustrative Embodiment 10. The apparatus of Illustrative Embodiment 9, wherein the acrylic adhesive tape is Polyethylene Foam Single Coated Medical Tape 1773.

Illustrative Embodiment 11. The apparatus of claim 9, wherein the silicone adhesive tape is Single-Coated Medical Nonwoven Tape with Hi-Tack Silicone Adhesive on Liner

Illustrative Embodiment 12. The apparatus of any of the preceding Illustrative Embodiments, wherein the skin contact layer is disposed on the skin-facing surface of the at least one electrode element.

Illustrative Embodiment 13. The apparatus of any one of Illustrative Embodiments 1-4, wherein the adhesive composite comprises a hydrogel.

Illustrative Embodiment 14. The apparatus of any one of Illustrative Embodiments 1-4, wherein the adhesive composite comprises: i) an acrylic polymer, or a silicone polymer, or a combination thereof; and ii) a hydrogel.

Illustrative Embodiment 15. A method comprising: positioning one or more electrode assemblies on a target site of a subject, the one or more electrode assemblies comprising: at least one electrode element having a skin-facing surface; a skin contact layer comprising a conductive adhesive composite, and an adhesive tape or bandage, wherein the at least one electrode element is electrically coupled to the skin contact layer.

Illustrative Embodiment 16. The method of Illustrative Embodiment 15, further comprising removing at least one of the one or more electrode assemblies from the target site of the subject after a period of time.

Illustrative Embodiment 17. The method of Illustrative Embodiment 16, wherein the removing comprises applying one or more of mineral oil, hexamethyldisiloxane, and a wipe comprising isopropyl alcohol, hydrotreated heavy naphtha, or (2-methoxymethylethoxy)propanol or combination thereof.

Illustrative Embodiment 18. The method of any one of Illustrative Embodiments 16-17, further comprising applying an alternating voltage between at least two electrode assemblies, thereby generating an alternating electric field, wherein applying the alternating voltage occurs prior to removing at least one of the electrode assemblies.

Illustrative Embodiment 19. The method of any one of Illustrative Embodiments 16-18, wherein the period of time is between 5 to 60 minutes, 1 to 24 hours, 1 to 7 days, 1 to 4 weeks, or 1 to 6 months.

Illustrative Embodiment 20. The method of any one of Illustrative Embodiments 16-18, further comprising, after removing at least one of the one or more electrode assemblies, applying a skin protectant agent to the target site.

Illustrative Embodiment 21. The method of Illustrative Embodiment 20, further comprising applying a skin protectant agent to the target site either: i) prior to positioning at least one or more electrode assemblies on a target site, or ii) after removing at least one of the one or more electrode assemblies.

Illustrative Embodiment 22. The method of any one of Illustrative Embodiments 20-21, wherein the skin protectant agent comprises one or more of a steroid, an alcohol, and a siloxane copolymer.

Illustrative Embodiment 23. The method of Illustrative Embodiment 22, wherein the steroid comprises betamethasone.

Illustrative Embodiment 24. The method of Illustrative Embodiment 22, wherein the alcohol comprises isopropyl alcohol.

Illustrative Embodiment 25. The method of Illustrative Embodiment 22, wherein the siloxane copolymer comprises a hexamethylsiloxane/acrylate copolymer.

Illustrative Embodiment 26. The method of any one of Illustrative Embodiments 22 and 24, wherein the alcohol is applied by a wipe.

Illustrative Embodiment 27. The method of Illustrative Embodiment 15, wherein the conductive adhesive composite comprises a hydrogel.

Illustrative Embodiment 28. The method of Illustrative Embodiment 27, wherein the conductive adhesive composite further comprises an acrylic polymer, or a silicone polymer, or a combination thereof.

Illustrative Embodiment 29. The method of Illustrative Embodiment 15, wherein the conductive adhesive composite comprises: a dielectric material; and conductive particles dispersed within the dielectric material.

Illustrative Embodiment 30. The method of Illustrative Embodiment 29, wherein at least a portion of the conductive particles define a conductive pathway through a thickness of the conductive adhesive composite.

Illustrative Embodiment 31. The method of any one of Illustrative Embodiments 29-30, wherein the dielectric material is an acrylic adhesive or a silicone adhesive, or a combination thereof.

Illustrative Embodiment 32. The method of any one of Illustrative Embodiments 29-31, wherein a hydrogel is not present between the target site and the skin-facing surface of the one or more electrodes.

Illustrative Embodiment 33. The method of Illustrative Embodiment 16, further comprising, after removing the at least one of the one or more electrode assemblies from the target site, re-positioning the at least one of the one or more electrode assemblies on the target site.

Illustrative Embodiment 34. The method of Illustrative Embodiment 33, further comprising, after re-positioning, removing the one or more electrode assemblies after a second period of time.

Illustrative Embodiment 35. The method of claim 33, further comprising applying a second alternating voltage between at least two electrode assemblies, thereby generating an alternating electric field, wherein applying the second alternating voltage occurs after re-positioning the at least one of the electrode assemblies and prior to removing the one or more electrode assemblies after a second period of time.

Illustrative Embodiment 36. The method of any one of Illustrative Embodiments 34 or 35, wherein the second period of time is between 5 to 60 minutes, 1 to 24 hours, 1 to 7 days, 1 to 4 weeks, or 1 to 6 months.

Illustrative Embodiment 37. The method of any one of Illustrative Embodiments 16-36 wherein, after removal of the one or more electrode assemblies, evaluating the target site for an adverse event.

Illustrative Embodiment 38. The method of Illustrative Embodiment 37, wherein the adverse event is contact dermatitis, pruritus, erosions, ulcers, folliculitis, or xerosis.

Illustrative Embodiment 39. The method of any one of Illustrative Embodiments 16-38, further comprising determining the ease of removing the one or more electrode assemblies.

Illustrative Embodiment 40. The method of any one of Illustrative Embodiments 16-39, wherein the conductive adhesive composite of the one or more electrode assemblies has a thickness ranging from about 30 µm to about 2000 µm.

Illustrative Embodiment 41. The method of any one of Illustrative Embodiments 16-40, wherein the conductive adhesive composite of one electrode assembly can be different from the conductive adhesive composite of another electrode assembly.

Illustrative Embodiment 42. The method of any one of claims 16-41, wherein the target site does not comprise hair.

Illustrative Embodiment 43. A kit comprising the apparatus of any one of Illustrative Embodiments 1-14.

Illustrative Embodiment 44. The kit of Illustrative Embodiment 43, further comprising a skin protectant agent.

Illustrative Embodiment 45. The kit of Illustrative Embodiments 43-44, further comprising instructions for carrying out one or more of the methods of claims 15-42.

We claim:

1. An apparatus comprising:
at least one electrode element having a skin-facing surface;
a skin contact layer comprising an adhesive composite; and
an adhesive tape or bandage.

2. The apparatus of claim 1, wherein the at least one electrode element is electrically coupled to the skin contact layer.

3. The apparatus of claim 1, wherein the adhesive composite is a conductive adhesive composite comprising:
a dielectric material; and
conductive particles dispersed within the dielectric material.

4. The apparatus of claim 3, wherein the dielectric material is an acrylic adhesive or a silicone adhesive, or a combination thereof.

5. The apparatus of claim 1, wherein the adhesive composite comprises a hydrogel.

6. The apparatus of claim 5, wherein the adhesive composite comprises:
an acrylic polymer, or a silicone polymer, or a combination thereof; and
a hydrogel.

7. A method comprising:
positioning one or more electrode assemblies on a target site of a subject, the one or more electrode assemblies comprising:
at least one electrode element having a skin-facing surface;
a skin contact layer comprising a conductive adhesive composite, and
an adhesive tape or bandage,
wherein the at least one electrode element is electrically coupled to the skin contact layer.

8. The method of claim 7, further comprising removing at least one of the one or more electrode assemblies from the target site of the subject after a period of time.

9. The method of claim 8, wherein the removing comprises applying one or more of mineral oil, hexamethyldisiloxane, and a wipe comprising isopropyl alcohol, hydrotreated heavy naphtha, or (2-methoxymethylethoxy)propanol or combination thereof.

10. The method of claim 9, further comprising applying an alternating voltage between at least two electrode assemblies, thereby generating an alternating electric field,
wherein applying the alternating voltage occurs prior to removing at least one of the electrode assemblies.

11. The method of claim 8, further comprising applying a skin protectant agent to the target site either:
prior to positioning at least one or more electrode assemblies on a target site, or
after removing at least one of the one or more electrode assemblies.

12. The method of claim 11, wherein the skin protectant agent comprises one or more of a steroid, an alcohol, and a siloxane copolymer.

13. The method of claim 12, wherein the steroid comprises betamethasone.

14. The method of claim 12, wherein the siloxane copolymer comprises a hexamethylsiloxane/acrylate copolymer.

15. The method of claim 11, wherein the conductive adhesive composite comprises a hydrogel.

16. The method of claim 15, wherein the conductive adhesive composite further comprises an acrylic polymer, or a silicone polymer, or a combination thereof.

17. The method of claim 8, wherein the conductive adhesive composite comprises:
a dielectric material; and
conductive particles dispersed within the dielectric material.

18. The method of claim 8, further comprising, after removing the at least one of the one or more electrode assemblies from the target site, re-positioning the at least one of the one or more electrode assemblies on the target site.

19. The method of claim 18, further comprising, after re-positioning, removing the one or more electrode assemblies after a second period of time.

20. The method of claim 18, further comprising applying a second alternating voltage between at least two electrode assemblies, thereby generating an alternating electric field, wherein applying the second alternating voltage occurs after re-positioning the at least one of the electrode assemblies and prior to removing the one or more electrode assemblies after a second period of time.
